(19) United States
(12) Patent Application Publication
(10) Pub. No.: US 2008/0087283 A1
(43) Pub. Date: Apr. 17, 2008

(54) APPARATUS FOR AND RELATED METHOD OF INHIBITING LEWIS ACID DEGRADATION IN A VAPORIZER

(75) Inventors: Keith R. Cromack, Gurnee, IL (US); Dennis A. Stephens, Mount Prospect, IL (US); Gowdahalli N. Subbarao, Mundelein, IL (US); Jeff Drajesk, Burlington, WI (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 11/873,216

(22) Filed: Oct. 16, 2007

Related U.S. Application Data
(60) Provisional application No. 60/829,612, filed on Oct. 16, 2006.

(51) Int. Cl. A61M 16/18

(52) U.S. Cl. 128/203.12

(57) ABSTRACT

The present invention relates to an anesthesia vaporizer, more particularly, a vaporizer for use in administering anesthesia wherein the vaporizer is designed to inhibit the degradation of anesthesia that is contained within the vaporizer. The invention further provides a method of preparing a vaporizer to inhibit the degradation of anesthesia.

### CROSS-REFERENCE TO RELATED APPLICATIONS

Not applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable.

### TECHNICAL FIELD

The present invention relates to an anesthesia vaporizer, more particularly, to a vaporizer for use in administering anesthesia wherein the vaporizer is designed to inhibit the degradation of anesthesia that is contained within the vaporizer. The invention further provides a method of preparing and/or treating a vaporizer to inhibit the degradation of anesthesia within the vaporizer.

### BACKGROUND OF THE INVENTION

During surgical and examination procedures patients can be administered anesthesia by various methods. One particular method includes the use of inhalation anesthesia wherein a liquid anesthesia is vaporized and mixed with various gases, such as oxygen and nitrous oxide. The liquid anesthesia is transformed into a vapor through the use of a vaporizer. The administration of liquid anesthesia must be closely monitored by physicians, i.e. anesthesiologist, in order to provide the correct dosage to the patient undergoing the particular procedure and ensure the patient's vitals are maintained within a certain range. Accordingly, the type of anesthesia, the overall effects of the anesthesia and the equipment administering the anesthesia must be maintained and monitored with the utmost precision and diligence.

Traditional vaporizers include a vaporizer chamber or reservoir wherein the liquid anesthesia is stored for administration to the patient. Depending on the type of surgical procedure being performed and the age or size of the patient, an anesthesiologist may have to fill the reservoir several times during the procedure, or conversely, the reservoir may contain enough liquid anesthesia to serve multiple patients and remain in the reservoir of the vaporizer for an extended period of time. Due to the fact that vaporizers are seldom allowed to run dry, some amount of anesthetic agent will be present in the vaporizer for most of its useful product life. The fact that the anesthetic agent may be maintained in a vaporizer over an extended period of time, adds in another variable that must be considered when determining the effectiveness and safety of the anesthesia being administered.

A number of different vaporizers are known and traditionally used for administering anesthesia by inhalation. Although the various vaporizers may be modified slightly depending on the types of chambers/reservoirs, control valves or connection system used, most vaporizers generally operate in the same fashion. In particular, most vaporizers contain a chamber or reservoir wherein the anesthetic agent is stored in liquid form prior to vaporizing the anesthesia for administration. Many vaporizers further include a wick assembly that assists in vaporizing the liquid anesthesia. The wick assemblies are designed to aid in the vaporization process and come into direct contact with the liquid anesthesia.

Anesthetic Agents

Inhalable anesthetics are typically volatile substances with relatively low boiling points and high vapor pressures. They can be flammable and explosive substances in both their liquid and vapor states. Further, inhalation of the vapor by health care personnel using them can cause drowsiness.

Therefore, such anesthetics must be safely handled in operating rooms in order to minimize the risk of inhalation by medical personnel as well as to minimize the risk of fire or explosion. Preferably, the anesthetic should be used in a way which will ensure that there is little or no release to the atmosphere at all stages of handling during normal surgical procedures.

Devices have been designed for the transfer of an anesthetic from a supply container to a vaporizer through a closed system that eliminates the escape of an anesthetic gas to the atmosphere. The devices are designed so that during set-up and disassembly procedures, a supply container of anesthetic is not open and exposed to the atmosphere in the operating room.

Due to the volatile nature of anesthetic agents and the desire to keep them contained, anesthetic agents are typically dispensed in single smaller batch container, i.e. 250 ml, and are readily adapted with a connector apparatus on the container that is readily received by the vaporizer. Devices have been designed for the transfer of an anesthetic from a supply container to a vaporizer through a closed system that minimizes the likelihood of the escape of an anesthetic gas to the atmosphere. The devices are designed so that during set-up and disassembly procedures, a supply container of anesthetic is not open and exposed to the atmosphere. Such systems that have been developed for connecting an anesthetic container to a vaporizer include the SECURITY LOCK™. Vapofill vaporizer connector and QUIK-FIL™ vaporizer system, each sold by Abbott Laboratories, Inc., One Abbott Park Road, Abbott Park, Ill. 60064-3500, U.S.A. Modifications made to container assemblies and connection systems include those provided in U.S. Pat. No. 5,505,236 entitled “Anesthetic vaporizer filling system” and U.S. Pat. No. 5,810,001 entitled “Anesthetic Transfer System”, both of which are incorporated herein by reference in their entirety. Overall, it is apparent that due to the volatile nature of anesthetic agents and the sensitivity to atmospheric environments, it is necessary to take great care to avoid unintended exposure of medical staff to the anesthesia when administering it. Therefore, the vaporizer is sealed system that has limited access to the external environment.

Degradation of Anesthetic Agents

Fluoroether compounds are commonly employed as anesthetic agents. Examples of fluoroether compounds used as anesthetic agents include sevoflurane (fluoromethyl-2,2,2-trifluoro-1-(trifluoromethyl)ethyl ether), enflurane ((±−)-2-chloro-1,1,2-trifluoroethyl difluoromethyl ether), isoflurane (1-chloro-2,2,2-trifluoroethyl difluoromethyl ether), methoxyflurane (2,2-dichloro-1,1-difluoroethyl methyl ether), and desflurane ((±−)-2-difluoromethyl 1,2,2,2-tetrafluoroethyl ether).

Although fluoroethers are excellent anesthetic agents, it has been discovered that some fluoroethers experience stability problems. More specifically, it has been determined that certain fluoroethers, such as sevoflurane, in the presence of one or more Lewis acids, degrade into several products including potentially toxic chemicals such as hydrofluoric acid. Hydrofluoric acid is toxic by ingestion and inhalation and is highly corrosive to skin and mucous membranes. Thereupon, the degradation of fluoroethers to chemicals such hydrofluoric acid is of great concern to the medical community.

Degradation of sevoflurane has been found to occur in glass containers. The degradation of sevoflurane in glass containers is believed to be activated by trace amounts of Lewis acids present in the container. One source of the Lewis acids can be aluminum oxides, which are a natural component of glass. When the glass wall becomes altered or etched in some manner, an aluminum oxide becomes exposed and comes into contact with the contents of the container. The Lewis acids then attack the sevoflurane and degrade it.

For example, when the sevoflurane is contacted with one or more Lewis acids in a glass container under anhydrous conditions, the Lewis acid initiates the degradation of sevoflurane to hydrofluoric acid and several degradation products. The degradation products of sevoflurane are hexafluoroisopropyl alcohol, methyleneglycol bishexafluoroisopropyl ether, dimethyleneglycol bishexafluoroisopropyl ether and methyleneglycol fluoromethyl hexafluoroisopropyl ether. The hydrofluoric acid proceeds to further attack the glass surface and expose more of the Lewis acid on the glass surface. This results in a cascading reaction with further degradation of sevoflurane. Methods of inhibiting degradation of fluoroether compositions have been developed by Abbott Laboratories, such as those provided in U.S. Pat. Nos. 5,990,176, 6,288,127, 6444,859, and 6,677492 and U.S. Ser. No. 10/606,821, all entitled “Fluoroether Compositions and Methods for Inhibiting their degradation in the presence of Lewis Acid”, all of which are incorporated herein by reference in their entirety. As these patents disclose, the amount of Lewis acid inhibitor necessary to inhibit the degradation of a fluoroether such as sevoflurane depends on several factors including temperature and type of materials to which the sevoflurane is exposed. Ultane® is a sevoflurane anesthetic agent manufactured by Abbott Laboratories, which contains at least 300 parts per million (ppm) and not more than 2,000 ppm of water.

Lewis acid degradation was first observed within the glass containers that were used to store the fluoroether compounds. In order to inhibit Lewis acid degradation that may occur in the glass containers, many steps have been taken including: modifying the container that the anesthetic agent is provided in, such as a non-oxidizing container (i.e. Polyethylene napthalate (PEN)) which is not a source of Lewis acids, and/or adding a Lewis acid inhibitor (such as water) to the anesthetic agent to react with Lewis acids and thereby inhibit degradation.

Examples of Lewis acids include common metal oxides which frequently are present on the metallic surfaces of manufacturing equipment, transport containers, and commonly used medical devices. Accordingly, even with the modifications of the anesthetic containers and formulas, exposure to medical devices and related equipment increases the likelihood of Lewis acid degradation of the anesthetic agents, in particular, fluoroethers, such as sevoflurane.

Current Vaporizer Systems

Current vaporizer systems including, but not limited to, Penlon Sigma Elite, Penlon Sigma Delta, Datex-Ohmeda Tec 5, Datex-Ohmeda Tec 7 and Drager Vapor 2000, are commonly used with fluoroether based anesthetic agents, in particular fluoroether sevoflurane. Throughout the operation of current vaporizers, anesthesia agents are provided within a reservoir of a vaporizer, vaporized and then administered to the patient through a breathing circuit. The reservoirs are refilled within the vaporizer units when necessary. Although most of the anesthesia may be used during the surgical procedure, some residual anesthesia remains within the reservoir of the vaporizer for extended periods of time due to the fact that vaporizers are not run dry. In general, it may be a number of years before the vaporizers are drained and/or cleaned depending on the duration of maintenance intervals. Moreover, vaporizers are designed to be used with one particular anesthetic agent; therefore it is been considered unnecessary to clean the residual anesthesia that remains within the reservoir after each use. Components of vaporizer systems are commonly made of oxidizing metals that provide Lewis acids. Such components include, but not limited to: an interface block, a fluid reservoir/chamber, a filler unit, the wick and/or wick assembly, control valves having fasteners, an observation window, flow compensation channels and any other components that would be a source of Lewis acids within the vaporizer system. Most of the current vaporizer components are comprised of oxidizing metals that include metals such as: brass, nickel, zinc, chromium, copper, stainless steel, iron or alloys of these and other oxidizing metals. It is known that electropolishing stainless steel will prevent oxidation, but due to the limitations of electropolishing, there are certain areas of the vaporizer that are difficult to access and can still be contributors of Lewis acids. It now has been found that residual anesthesia remaining within the reservoir comes in contact with many of these vaporizer components comprised of oxidizing metals and is degraded by that Lewis acid within the vaporizer itself.

Accordingly, the inventors have discovered a need to modify the current existing vaporizers to minimize the risks associated with Lewis acid degradation based on the exposure of the anesthetic agents to the internal vaporizer components that contribute Lewis acids. The degradation of anesthesia agents ultimately impacts the safety of the patients and accordingly needs to be addressed.

### SUMMARY OF THE INVENTION

The present invention provides an apparatus having components made of non-oxidizing metals or materials, thereby inhibiting degradation of fluoroethers, in particular, sevoflurane that occurs upon exposure to a Lewis acid. More particularly, the present invention provides a vaporizer having components made of non-oxidizing materials or metals including, but not limited to, platinum, gold, or any other metal having similar properties. In addition, the present invention provides a vaporizer comprising an observation window coated with a hard coat polymer or made of a hard coat polymer. In a preferred embodiment, the hard coat or hard coat polymer is selected from the group consisting of polyacrylic, polycarbonate, and polymethacrylic polymer coatings

The present invention further provides a vaporizer whose components can be plated with a non-oxidizing metal thereby reducing the amount of Lewis acids exposed to the anesthetic agent within the vaporizer and related components. Preferably, the non-oxidizing metal is provided through plating technology, such as, for example, chemical plating, electrochemical plating, vapor deposition and plasma deposition. The present invention may further be designed to increase the efficiency of coating (i.e. rounded and square corners of vaporizer components). The present invention further provides a wick assembly or other vaporizer components having non-oxidizing materials and/or non-water absorbing materials to inhibit degradation of the anesthetic agent. A non-oxidizing material may be a polyamide such as nylon. Overall, the present invention provides a vaporizer and related components that inhibit Lewis acid degradation of an anesthesia agent that occurs within a vaporizer.

The present invention further provides a method for treating a vaporizer with a Lewis acid inhibitor to inhibit Lewis acid degradation of an anesthesia agent within the vaporizer. In one particular embodiment, the present invention provides a method of exposing the components of the vaporizer with a Lewis acid inhibitor, e.g. water, prior to adding the anesethetic agent to the vaporizer. The present invention further provides a method of treating vaporizer components by nebulizing a Lewis acid inhibitor composition and adding the vapor of the Lewis acid inhibitor to the internal compartment of the vaporizer. The nebulizing of the vaporizer may occur intermittently and either before, during or after the administering of anesthesia to a patient. The present invention further provides a vaporizer, which includes a Lewis acid inhibitor source to inhibit degradation. Overall, the treatment of a vaporizer with a Lewis Acid inhibitor will aid in the inhibition of Lewis Acid degradation of an anesthesia agent that can occur during exposure of vaporizer components to anesthetic agents, such as fluoroethers.

The present invention further provides for a combination of methods to inhibit Lewis acid degradation of an anesthesia agent within a vaporizer. In particular, the present invention provides a vaporizer having components made of non-oxidizing metals and/or materials that have been treated with or exposed to Lewis acid inhibitors.

Numerous other advantages and features of the present invention will become readily apparent from the following detailed description of the invention, from the claims, and from the accompanying figures and tables.

### FIGURES

FIG. 1 is front perspective view of an exemplary vaporizer known by those skilled in the art;

FIG. 2 is a schematic view of the internal components of a vaporizer;

FIG. 3 is a wick assembly to be incorporated within vaporizer as shown in FIG. 2;

FIG. 4 is a diagrammatic illustration of one embodiment of the present invention;

FIG. 5 is a perspective view of an alternate embodiment of the present invention;

FIG. 6 is a perspective view of another alternate embodiment of the present invention; and

FIG. 7 is a perspective view of another alternate embodiment of the present invention as provided in FIG. 6.

It should be appreciated that the drawings are for illustrative purposes only.

### DETAILED DESCRIPTION OF THE INVENTION

The present invention provides a novel vaporizer and method of treating a vaporizer to inhibit Lewis acid degradation of the anesthetic agents contained within the vaporizer.

Anesthetic agents are typically not administered directly from the container to the patient. Rather, an anesthetic agent is poured into a vaporizer, which is connected to an anesthesia machine including a breathing circuit. This machine delivers a titrated amount of drug to the patient by controlling the flow of oxygen, air, and nitrous oxide that have circulated through the vaporizer. Accordingly, the concentration of drug given to a patient should be individualized based on the patient's response.

A vaporizer includes a fluid reservoir containing the liquid anesthetic agent and a wick that helps to volatize the agent. Vaporizers are designed to administer only one kind of anesthetic. When a vaporizer is engaged, the anesthetic agent flows continuously throughout the breathing circuit of the anesthesia machine, including all of the valves and conduits in the machine itself.

Vaporizers contain substantial amounts of metal that are in direct contact with the anesthetic agent. They also contain bi-metallic valves that stabilize the flow of the agent despite small fluctuations in the temperature inside the vaporizers. When fluoroether anesthetic agents are placed inside a vaporizer, the anesthesia may be exposed to these metals for a prolonged period of time.

The components of vaporizers are generally known to those skilled in the art. The structure of general vaporizers are described and further set forth in U.S. Pat. No. 6,138,672 entitled “Arrangement in Anesthesia Vaporizer”, U.S. Pat. No. 6,443,150 entitled “Anaesthetic Vaporizer”, U.S. Pat. No. 6,230,666 entitled “Vaporizer”, and U.S. Pat. No. 5,915,427 entitled “Anesthetic Vaporizer Draining System”, all of which are incorporated by reference herein in their entirety and are exemplary in nature of vaporizers used in the industry. Vaporizers used in the industry include, but are not limited to, Penlon Sigma Elite, Penlon Sigma Delta, Datex-Ohmeda Tec 5, Datex-Ohmeda Tec 7 and Drager Vapor 2000. All of these vaporizers are only exemplary in nature and the present invention may be applied to any vaporizer used in the industry. Accordingly, the structure and design of vaporizers are generally known by those in the industry.

Generally, vaporizers in the industry are comprised of aluminum, stainless steel, or other oxidizing metals, such as brass, copper, nickel, iron, and alloys which include these metals. Accordingly, it has been discovered that these materials interact with anesthetic agents resulting in degradation.

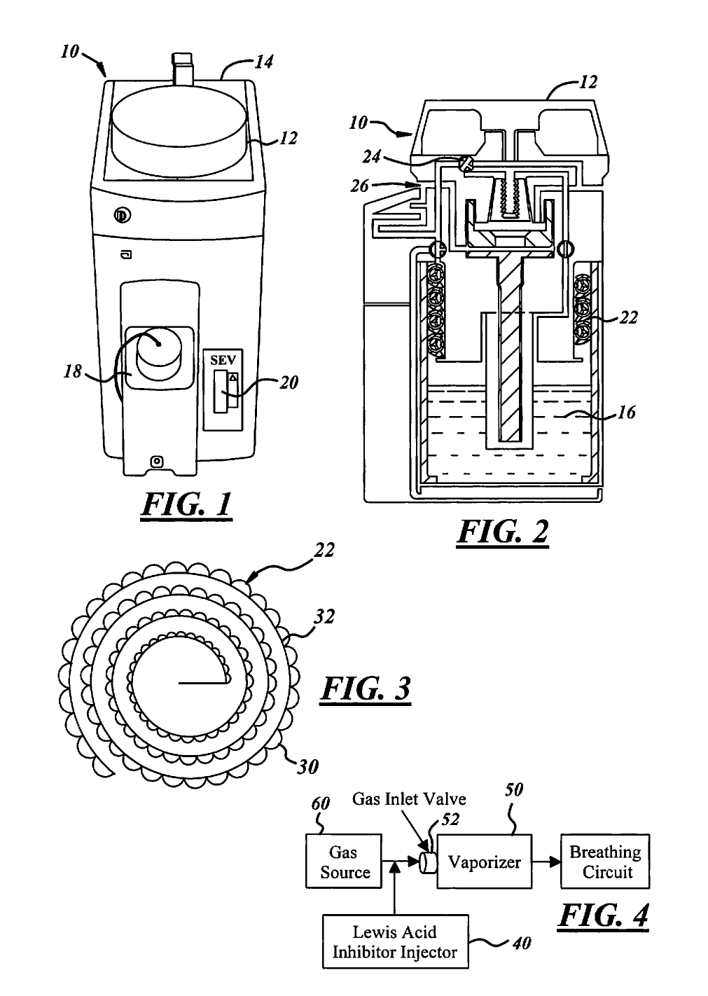

As can be seen in FIGS. 1 and 2, which is exemplary in nature of a common vaporizer used in practice, vaporizer (10) is defined by an outer housing (12) that further provides components including, but not limited to, an interface block (14), a fluid reservoir (16), a filler unit (18), an observation window (20), a wick assembly (22) and related valves (24) and ports (26). The valves (24) are provided for regulating the operation and related conditions of the vaporizer, such as vapor flow, temperature, and drug regulation. Ports (26) may be provided for connecting the vaporizer (10) to other breathing circuit components and anesthesia units for administering anesthesia to a patient. The structure of each of these components are generally known in the industry by one of ordinary skill in the art. In addition, other components of the vaporizer, which are not shown, are known in the industry and are necessary for general operation of the vaporizer. Such components are incorporated in this present invention in order to provide a fully operable vaporizer.

In general, the vaporizer (10) is defined by an outer housing (12) that provides the internal components of the vaporizer (10). The internal structure of a vaporizer is well know in the industry and the provided Figures are exemplary in nature. Housing (12) of vaporizer (10) provides a fluid reservoir (12) wherein the anesthesia is held and maintained during the operation of the vaporizer. Following the filling of vaporizer (10) through the filler unit (18), the anesthesia is disposed within fluid reservoir (12). The level of anesthesia within the fluid reservoir (12) may be determined by viewing the level of anesthesia indicated by the observation window (20). The wick assembly (22) is further provided within the housing (12) of the vaporizer (10) and is partially exposed to the anesthesia that is held within the fluid reservoir (16). During operation of vaporizer (10), the wick assembly (22) absorbs anesthesia from the fluid reservoir (16) and facilitates evaporation of the anesthesia. Gases, such as oxygen, compressed air, and/or nitrous oxide, travel through the vaporizer, over the wick assembly (22) thereby absorbing the anesthesia vapor created by the wick assembly (22). The anesthesia vapor then travels throughout the vaporizer (10) and comes into contact with valves (24) and ports (26) disposed through the vaporizer (10). Additional components, such as o-rings and gaskets are provided throughout the vaporizer and are exposed to the anesthetic fluid and vapor prior to being administered to the patient.

Most of the components of vaporizers come in direct contact with anesthetic agents, such as sevoflurane, during the vaporizing process. Some components, in particular, are more often in direct contact with the fluoroether anesthesia compounds during the filling, storage and vaporization process that occurs within the vaporizer. As previously discussed above, such components include the filler unit (18) (also referred to as the filler port or filler port shoe) wherein anesthesia is put into the vaporizer (10), the observation window (20) (also referred to as the sight glass) wherein one can observe the level of anesthetic within the vaporizer; the fluid reservoir (16) (also referred to as the container) that stores the liquid anesthesia; and the wick assembly (22) (also referred to as the “wick”) that assists in the vaporization of the anesthesia. Due to the fact that these components are more commonly in direct contact with the fluoroether anesthesia, these components are more likely to contribute potential Lewis acids that result in degradation of the fluoroether anesthesia.

Accordingly, in order to inhibit Lewis acid degradation, the vaporizer of the present invention provides components made out of non-oxidizing metals or materials. Furthermore, portions of various components may be made of non-oxidizing materials or metals. The non-oxidizing metals of the present invention are not a source of Lewis acids and therefore do not result in degradation of the anesthetic agents. Such non-oxidizing materials preferably can include, but are not limited to, platinum, gold, or any combinations thereof. Therefore the present invention includes a fluid reservoir (16), a filler unit (18), an observation window (20) and wick assembly (22) that is comprised of nonoxidizing materials, in particular, non-oxidizing metals. Furthermore, select portions of these components, in particular those that are directly exposed to liquid anesthesia for an extended period of time, may be made of non-oxidizing materials, preferably non-oxidizing metals. In particular, any portions of the vaporizer that are exposed to liquid anesthesia and are not easily electropolished are key sources of Lewis acid. These portions include areas surrounding the observation window (20), within the filler unit (18), and various channels throughout the vaporizer that enable vapor flow.

An alternate embodiment of the present invention provides components of a vaporizer that have undergone a plating process or have been coated with a non-oxidizing metal including, but not limited to platinum, gold, or any other non-oxidizing metals or combinations thereof. Due to the expense associated with making components of solid platinum and/or gold, it is more economically feasible to plate a component(s) of a vaporizer with a non-oxidizing metal thereby reducing the Lewis acids which may contribute to or cause degradation. A further embodiment includes components that have undergone vapor deposition of non-oxidizing metals, such as gold, platinum, or any other non-oxidizing metal having similar properties. Another type of coating method includes the use of plasma deposition to deposit non-oxidizing metals on the surfaces of the vaporizer components. As a result, components of the vaporizer that are coated with non-oxidizing metals do not provide Lewis acids to the fluoroether compounds. Accordingly, vaporizers having non-oxidizing metal components inhibit the Lewis acid degradation of an anesthesia agent that occurs within traditional vaporizers. Each of these plating or deposition processes are generally known in the art and are performed in accordance with standard procedures. Each of the components may be plated or coated with a non-oxidizing metal coating either in their entirety or only a portion of the component that may be exposed to anesthesia. The portions of the components that would preferably be coated with a non-oxidizing metal include the lower portion of the fluid reservoir, portions of the filler port, portions of the wick assembly and any other components directly exposed to liquid anesthesia.

There are several well known techniques for creating non-oxidizing metal coatings on surfaces, in particular, chemical plating or electrochemical plating. In chemical plating the metal to be deposited is in the form of a simple salt or a soluble complex compound in an aqueous solution. A reducing agent is needed to produce metal ions under certain well defined conditions of temperature and pH such that a deposit forms. In general the thickness of the deposited layer is uniform and the technique is very well adapted to plating in recesses or on the internal surface of small tubes. Electrochemical plating, often referred to as galvanic plating, is a more polyvalent technology. The part to be plated is negatively polarised in an aqueous solution containing either simple salts or complex compounds. Electrochemical reactions due to the electrical current produce metallic ions under certain conditions of temperature and pH. By proper design of the electrodes it is possible to produce quite uniform layers on complex geometries.

Several alternative technologies exist to coat a substrate with metal without using electrolytic solutions or plating baths. These include: Vapor Deposition (Ion plating, Ion implantation Sputtering and sputter deposition, Laser surface alloying), Chemical Vapor Deposition, and Thermal Spray Coating.

An alternate embodiment of the present invention provides vaporizer components coated in a hard coat, such as a polyacrylic, polycarbonate, or polymethacrylic polymer coating. The hard coat forms a nonporous robust barrier to inhibit Lewis acid degradation of an anesthesia agent within the vaporizer. The components of the vaporizer may be coated in their entirety or only part of the components may be coated. Because of the wide variety and complexity of parts which require coatings, numerous application methods are now in use. The coating of minute junctions or the application to huge motor rotors, has necessitated the development of ingenious and varied deposition and polymerization techniques.

There are several basic methods of application. Spray coating is the most widely used method of applying coatings to parts. Spraying, includes not only the commonly used compressed-air vaporization technique, but also airless pressure spray, hot spray, hot-vapor impelled spray, electrostatic spray, dry-powder resin. Other methods include dip coating, spin coating, roller coating, brush coating, electrocoating, and vacuum deposition. The final process may further include a crosslinking step to create a hard coat.

Each component within a vaporizer that comes in contact with an fluoroether anesthetic agent can potentially initiate Lewis acid degradation. Accordingly, each of the components can be made of a non-oxidizing metal, hard coat polymer coating or combinations thereof. Furthermore, each of the components may be coated either in their entirety or partially to inhibit degradation.

As previously suggested, different components of a vaporizer have various levels of contact with fluoroether compounds that are used as anesthetic agents. In traditional vaporizers, the interface block that connects the vaporizer to the anesthesia machine is made of stainless steel or anodized aluminum, and typically includes fluorocarbon O-rings for sealing and stainless steel fasteners for mechanical assembly. Vaporized sevoflurane comes into contact with these components as gases containing sevoflurane flow throughout the inner circuit of the vaporizer and anesthesia machine. Accordingly, Lewis acid degradation of an anesthesia agent is inhibited when each of these components include non-oxidizing metals, materials or combinations thereof. Furthermore, the entire interface can be made of these materials or only a portion of the interface block that comes most often comes into contact with the fluoroether compounds may be made of the non-oxidizing metals, polyacrylic polymer coating, or combinations thereof.

As previously discussed and described above, the fluid reservoir (16) stores the liquid anesthetic and is typically made of brass, aluminum, or stainless steel. In addition, the filler unit (18) connected to the reservoir (16) is also made of brass or stainless steel. As discussed above, liquid sevoflurane can remain in contact with these potential Lewis acid sources for extended periods of time. As a result, it is beneficial to create a reservoir that is comprised of non-oxidizing metals, a coating containing non-oxidizing metals, a coating of polyacrylic polymers, or combinations thereof. The filler unit is typically adjacent to the observation window (20) (also referred to as the sight glass). The observation window (20) is comprised of a glass or plastic window that allows the level of liquid anesthetic to be seen and provides an observation area to view the anesthesia levels within the vaporizer. This window may be in direct contact with liquid sevoflurane for an extended period of time. In addition, the window is frequently attached with stainless steel fasteners that may come into direct contact with either the liquid or vaporized anesthetic. As previously stated, it was determined that Lewis acid degradation was occurring in glass bottles that contained the fluoroether compounds. Accordingly, the glass of the observation window (20) would have some sort of Lewis acid degradation effect. Due to the nature of the observation window (20), it may be most feasible to coat the glass with a hard coat (as previously described) or to replace the glass of the observation window with hard coat material in order to inhibit Lewis acid degradation.

Another component that facilitates Lewis acid degradation in a vaporizer (10), as seen in FIGS. 2 and 3, is the wick assembly (22) that is incorporated with the vaporizer (10). The wick assembly (22) allows a larger surface of liquid sevoflurane to be presented to the circulating gases and is traditionally supported by a large metal structure (e.g. a backing component/plate) composed of copper or stainless steel. As previously discussed, the wick assembly (22) is exposed to both the liquid sevoflurane absorbed by the wick assembly (22) and to the vaporized sevoflurane being absorbed and circulated through the reservoir (16). Various wick assemblies are generally known in the art and have been discussed in more detail as provided in U.S. Pat. No. 4,774,032 entitled “Vaporizers and Wick Assemblies therefore” and incorporated herein by reference in its entirety.

One embodiment of the present invention further provides a wick assembly (22) that includes a wick (30) and optionally a backing member (32) that are both made of non-oxidizing materials. Common wick assemblies are comprised of cotton, oxidizing metals or other types of polymer materials. The wick assembly (22) of the present invention, as shown in FIG. 3, is comprised of a wick (30) and a backing member (32) that includes non-oxidizing metals or other materials. In particular the wick may be comprised of a polymer material which is supported by a backing of non-oxidizing metal. The non-oxidizing metals include gold, platinum, and any other non-oxidizing metal having similar properties. The backing member (32) of the wick assembly (22) may be optionally provided, therefore the wick (30) may be provided as the sole component of the assembly (22).

The present invention further provides a wick (30) made of a low-water absorbing, such as a polyamide or polyester material or non-water absorbing material which may include, hydrophobic polymers such as polyalkylacrylates, polydienes, polyolefins, polysiloxanes, and polypyridines. The wick assembly is partially submerged in the anesthesia. The small pores or capillaries within the wick act to draw up the anesthesia agent onto the high surface area wick to enhance evaporation rates. The presence of non-oxidizing materials within the wick assembly results in inhibition of degradation.

In addition to the above components, the flow of the gas through the vaporizer is controlled by valves (24) that typically include materials such as copper-zinc brass. In addition “free-machining” brass, which contains lead for easier machining during fabrication, is also at times present within the housing of the vaporizer. Each of these contribute Lewis acids and can affect degradation. Traditional vaporizers include valves that are typically brass or stainless steel, with Teflon or other fluorocarbon O-rings and seals. Many of the internal seals are “spring-energized,” or equipped with internal stainless steel springs to provide sealing pressure. Stainless steel fasteners are also typically used to connect these various parts. All of these potential Lewis acid sources are exposed to vaporized sevoflurane in the currently existing vaporizer systems. Accordingly, there is a need to provide a vaporizer that has non-oxidizing parts for each of these related components. Furthermore, non-water absorbing parts may be included in place of any components that have a tendency to absorb water. Accordingly, the present invention further provides an embodiment wherein water absorbing components of the vaporizer, are replaced with non-water absorbing components, such as (and for exemplary purposes) rubber o-rings are replaced with indium gaskets or hydrophobic polymers such as polyalkylacrylates, polydienes, polyolefins, polysiloxanes, polystyrenes, and polypyridine. One area in particular where a hydrophobic o-ring or seal arrangement would be beneficial is the area/seal around the observation window.

Lastly, anesthesia and vaporizer machines typically contain other mechanisms, like bi-metallic valves, to maintain consistent temperature and drug concentrations. These materials are similar to other metering components, and, again, come in contact with vaporized sevoflurane. Additional components, such as these, can be made of non-oxidizing materials in order to prevent the contribution of Lewis acids to the vaporizer system.

Testing has revealed that vaporizers currently used for sevoflurane delivery are sources of potential Lewis acids. The total surface area of potential Lewis acid formers in contact with sevoflurane in each of three new vaporizers is listed in Table 1:

TABLE 1
Surface Area of Potential Lewis Acids in New Vaporizers

| | Surface Area of | Surface Area of |
|---|---|---|
| | Potential LA | Potential LA |
| | Exposed to Liquid | Exposed to Vapor |
| Vaporizer | (cm²) | (cm²) |
| Datex-Ohmeda Tec 7 (GE) | 183 | 2189 |
| Drager Vapor 2000 | 917 | 481 |
| Penlon Sigma Elite | 400 | 429 |

The total surface area of potential Lewis Acid formers in contact with sevoflurane in each of three used vaporizers is listed in Table 2:

TABLE 2
Surface Area of Potential Lewis Acids in Used Vaporizers

| | Surface Area of | Surface Area of |
|---|---|---|
| | Potential LA | Potential LA |
| | Exposed to Liquid | Exposed to Vapor |
| Vaporizer | (cm²) | (cm²) |
| Datex-Ohmeda Tec 7 (GE) | 207 | 189 |
| Drager Vapor 2000 | 917 | 481 |
| Penlon Sigma Elite | 409 | 429 |

Another embodiment of the present invention include the pretreatment or intermittent treatment of a vaporizer either prior to adding the fluoroether anesthesia agent or during the administering of a fluoroether anesthesia agent in order to increase the presence of Lewis acid inhibitors within the vaporizer to inhibit Lewis acid degradation. Any Lewis acid inhibitor may be used to inhibit degradation within a vaporizer. Such Lewis acid inhibitors include, but are not limited to, water, hydroxytoluene, methylparaben, propylparaben, propofol, and thymol. Water is the most preferred Lewis acid inhibitor.

Various methods may be used to achieve this aspect of the present invention. Such methods include incorporating the use of a Lewis acid inhibitor injector, such as a nebulizer, with a vaporizer. Nebulizers are generally known in the art. Nebulizers are traditionally used to administer small doses of liquid medication to patients, typically for respiratory ailments, in a vapor form for inhalation. In addition, nebulizers have been incorporated within anesthesia breathing circuits to add water vapor to the breathing circuit for the purpose of making a patient more comfortable due to the overall dryness of the circuit when administering anesthesia to a patient. Various nebulizing systems include, but are not limited, a Pall Biomedical Products, Co.; Easthill, N.Y. and a Fischer Paykel model MR630 respiratory circuit and anesthesia circuit humidifier. The incorporation of nebulizers into anesthesia breathing circuits is further discussed in U.S. Pat. No. 6,550,476 and US Patent Appl No. 2006/0012057, both of which are incorporated by reference herein in their entirety. The administration of oxygen gas to a patient has a drying effect on the patient's mucous membranes. In current breathing circuits, nebulizers are added downstream from a vaporizer, such as at the patient's mouthpiece or in combination with the carbon dioxide absorber that is used in rebreathing circuits. The nebulizer is provided in current breathing circuits to increase the moisture content of the oxygen that the patient is breathing. Based on the current design of the breathing circuits and the location of the nebulizer with the circuits, moisture provided by the nebulizer cannot get into the vaporizer.

The present invention provides a vaporizer having a Lewis acid inhibitor source incorporated with(in) the vaporizer. In accordance with the present invention, a Lewis acid inhibitor injector, such as a nebulizer, is positioned between the oxygen source that is providing the oxygen gas entering the vaporizer and the vaporizer itself. Accordingly, the vaporizer can be retrofitted to accommodate an adapter that would allow the port on the vaporizer that receives the oxygen gas from an external gas source, to receive a connector for a nebulizer that would provide vapor of a Lewis acid inhibitor, preferably water. The gas source may include various types of gases used during surgical procedures, including oxygen, compressed air and nitrous oxide. The vapor of the Lewis acid inhibitor will mix with the oxygen gas entering the vaporizer and consequently the anesthesia agent that is provided within the reservoir of the vaporizer. By adding vapor of a Lewis acid inhibitor (such as water vapor), any Lewis acid degradation that occurs within the vaporizer is inhibited based on the newly added presence of the Lewis acid inhibitor through the nebulizer. In accordance with this embodiment, a separate nebulizer may be added to a preexisting vaporizer through the adaptation of various ports and valves. As can be seen in FIGS. 4 and 5, the Lewis acid inhibitor injector (40) can be integrated with a vaporizer (50), such as those commercially available on the market and previously discussed, therein combining the vapor of a Lewis acid inhibitor with gas provided from the gas source (60) throughout the gas inlet valve (52).

In another embodiment of the present invention, a nebulizer is physically integrated within a vaporizer therein providing a single unit that maintains a certain moisture content within the vaporizer, wherein the moisture content is sufficient for inhibiting degradation.

The amount of nebulized Lewis acid inhibitor effective to inhibit degradation will depend on the composition and/or type of vaporizer (e.g. the number and types of components and component parts which may be sources of Lewis acids) as well as how much Lewis acid inhibitor is present in the sevoflurane compound. For example, it is understood that a vaporizer which receives a sevoflurane product having a water content of e.g. 400 ppm water to 500 ppm water will require less nebulized Lewis acid inhibitor than an identical vaporizer that receives a sevoflurane product having a water content of e.g. 20 ppm, 60 ppm, or 100 ppm. Also by way of example, a vaporizer that has more non-oxidizable components than a second vaporizer will require less nebulized Lewis acid inhibitor than one with many oxidizable components. “Degradation” is a term well understood by organic chemists and means a compound being broken down into other, generally unwanted, compounds. As used herein, an anesthetic composition, such as sevoflurane, is considered to be “degraded” when the byproducts formed from the breakdown of the sevoflurane molecule by the Lewis acids exceed a certain acceptable level of impurities. Methods of determining whether sevoflurane degradation has been inhibited are well known to those of ordinary skill in the art and can be determined easily using routine methodologies. The nebulizer plus vaporizer could include controls to regulate the amount of Lewis acid inhibitor being generated by the nebulizer and incorporated within the vaporizer. In addition, a gauge can be added, in order to detect the levels of the Lewis acid inhibitor within the vaporizer.

In order to maintain the Lewis acid inhibitor levels within a vaporizer to an effective amount to inhibit Lewis acid degradation, the Lewis acid inhibitor can be nebulized intermittently depending on the conditions of the vaporizer and atmospheric conditions that would overall impact the vaporizer conditions.

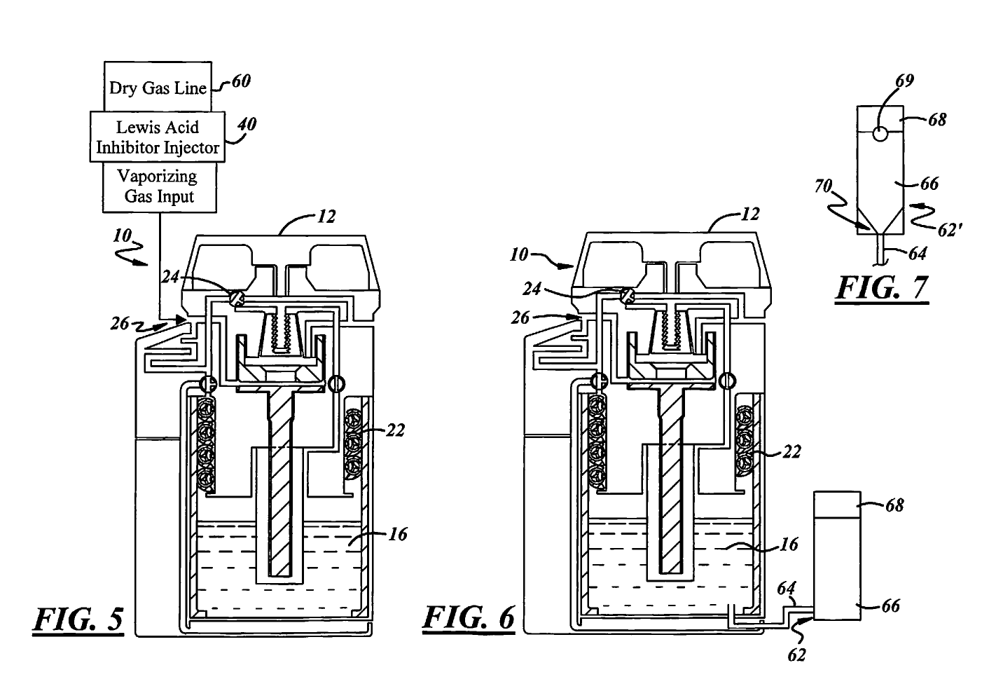

FIG. 6 further provides an alternate embodiment of the present invention, wherein a secondary vaporizer reservoir (62) is coupled to the primary vaporizer reservoir (16) by a connector (64). The secondary vaporizer reservoir (62) is provided with an anesthetic agent (66), such as sevoflurane, and further includes a Lewis acid inhibitor (68), such as water. The secondary vaporizer reservoir (62) is connected to the reservoir (16) of the vaporizer (10) by connector (64). The connector (64) may be in the form of tubing having a connector flow valve or any other type of fluid connection used in the industry. A current vaporizer may be modified to be coupled with an secondary vaporizer reservoir (62) or a new vaporizer may have the secondary vaporizer reservoir (62) integrated within the vaporizer. As can be seen in FIG. 6, the Lewis acid inhibitor (68) is less dense than the anesthetic agent (66). Therefore, the two liquids are separated based on densities. Due to the u-tube design of the connector (64), the Lewis acid inhibitor will never go within the vaporizer reservoir. The presence of the Lewis acid inhibitor causes the inhibitor to exist in equilibrium with the anesthetic agent.

FIG. 7 further provides an alternate embodiment of secondary vaporizer reservoir (62′) wherein one end 70 of the reservoir (62′) is conical in design and provides an opening for allowing the anesthetic agent (68) to flow into the connector (64). A float (69) is further provided within the reservoir (62′). The float has a density greater than the Lewis acid inhibitor (68) and less than the anesthetic agent (66). As the level of anesthetic agent is reduced and transferred into the connector (64) to ultimately reservoir (16), the float (69) moves towards end 70. Ultimately, float (69) will engage the opening of conical shaped end 70 and prevent the Lewis acid inhibitor from entering the reservoir (16) of the vaporizer.

Other methods of treating vaporizer components with a Lewis acid inhibitor, include, rinsing the reservoir with a Lewis acid inhibitor prior to adding new anesthetic agent and between administering of anesthesia to a patient. Any other method used to add a Lewis acid inhibitor, either in liquid or vapor form, can be applied to the present invention in order to expose the vaporizer to Lewis acid inhibitors and ultimately inhibit Lewis acid degradation.

Finally, the present invention further provides the ability to combine the above-mentioned invention to provide a vaporizer that inhibits Lewis acid degradation. Accordingly a vaporizer that includes components either made of or coated with non-oxidizing metals would be treated with Lewis acid inhibitor to inhibit degradation from occurring within the vaporizer, according to the standards and embodiments described above.

What is claimed is:

1. A vaporizer for inhibiting degradation of an anesthetic agent by a Lewis acid within said vaporizer, said vaporizer comprising a housing enclosing a fluid reservoir and a wick assembly, said fluid reservoir and wick assembly being comprised of a non-oxidizing material.

2. The vaporizer according to claim 1 wherein said non-oxidizing material is a non-oxidizing metal.

3. The vaporizer according to claim 2 wherein said non-oxidizing metal is selected from the group consisting of gold, platinum, and combinations thereof.

4. The vaporizer according to claim 2 wherein a portion of the fluid reservoir is comprised of a non-oxidizing metal.

5. The vaporizer according to claim 1 further comprising an observation window coated with a hard coat polymer.

6. The vaporizer according to claim 5 wherein said hard coat polymer is selected from the group consisting of polyacrylic, polycarbonate, and polymethacrylic polymer coatings.

7. The vaporizer according to claim 1 further comprising an observation window made from a hard coat polymer.

8. The vaporizer according to claim 7 wherein said hard coat polymer is selected from the group consisting of polyacrylic, polycarbonate, and polymethacrylic polymer coatings.

9. The vaporizer according to claim 1 further comprising a filler unit comprised of a non-oxidizing metal.

10. The vaporizer according to claim 1 wherein said wick assembly comprises a wick and a backing member wherein said wick and backing member are comprised of a non-oxidizing material.

11. The vaporizer according to claim 10 wherein said non-oxidizing material is a low-water absorbing or non-water absorbing material.

12. The vaporizer according to claim 1wherein said non-oxidizing material is a low-water absorbing material and said low-water absorbing material is a polyamide or a polyester.

13. The vaporizer according to claim 12 wherein said non-oxidizing material is a non-water absorbing material and said non-water absorbing material is selected from the group consisting of polyalkylacrylates, polydienes, polyolefins, polysiloxanes and polypyridines.

14. The vaporizer according to claim 2 wherein said non-oxidizing metal is provided through plating technology.

15. The vaporizer according to claim 14 wherein said plating technology is selected from the group consisting of chemical plating, electrochemical plating, vapor deposition and plasma deposition.

16. A method of inhibiting degradation of an anesthetic agent by a Lewis acid within a vaporizer comprising the steps of:
coupling a Lewis acid inhibitor injector to an inlet gas valve of a vaporizer; and
injecting a Lewis acid inhibitor into a vaporizer to prevent said degradation of said anesthetic agent.

17. The method according to claim 16 further comprising the step of maintaining a predetermined level of said Lewis acid inhibitor within said vaporizer.

18. The method according to claim 16 wherein said Lewis acid inhibitor injector is a nebulizer.

19. The method according to claim 19 wherein said Lewis acid inhibitor is water vapor.

20. A vaporizer comprising an integrated Lewis acid inhibitor source for supplying a Lewis acid inhibitor into the vaporizer system to inhibit degradation of an anesthetic agent by a Lewis acid within the vaporizer.

21. The vaporizer according to claim 21 wherein said Lewis acid inhibitor source is a Lewis acid inhibitor injector.

22. The vaporizer according to claim 22 wherein said Lewis acid inhibitor injector is a nebulizer.

23. The vaporizer according to claim 21 wherein said Lewis acid inhibitor source is a secondary reservoir connected to a primary reservoir wherein said Lewis acid inhibitor floats on said anesthetic agent.

24. The vaporizer according to claim 21 wherein said Lewis acid inhibitor is selected from the group consisting of water, hydroxytoluene, methylparaben, propylparaben, propofol, and thymol.

25. The vaporizer according to claim 26 wherein said Lewis acid inhibitor is water.
